(19) United States
(12) Patent Application Publication
(10) Pub. No.: US 2008/0234691 A1
(43) Pub. Date: Sep. 25, 2008

(54) Flex-Rod, Curvature-Adaptable

(76) Inventor: Helmut Schwab, Princeton, NJ (US)

Correspondence Address: HELMUT SCHWAB, 61 WESTCOTT ROAD, PRINCETON, NJ, 08540, US

(21) Appl. No.: 11/677,568

(22) Filed: Feb. 21, 2007

(51) Int. Cl. A61B 17/70; A61B 17/58

(52) U.S. Cl. 606/100

(57) ABSTRACT

The design of a rod is disclosed that can serve in general structural applications or as an orthopedic spinal implant to retain formation or stimulate the reduction of deformity as occurring in scoliosis and kyphosis. Different from common solid rod implants, the here-disclosed rod is curvature-adaptable in three dimensions to the actual or desired curvature or shape of the spine and can be provided with elasticity in order to serve as support or agent for curvature correction. The curvature adaptation of the proposed rod can be done with minimal force before or after implantation by means of the simple rotation of always only one in a pair of wedge shaped washers inserted between rod segments while leaving full mechanical strength or elasticity to the rod after curvature adaptation. The rotation of the washers can be done by various means, either once before implant or subsequently in the implanted state.

### SUMMARY

A rod is segmented and contains wedge-shaped washers between straight rod segments with either a central internal wire providing the connection or with interlocking surface features between the rod elements. The wedge-shaped washers are always arranged in pairs (or multiples thereof) of equally angled washers to permit zero rod curvature when staying in an “opposed rotation” arrangement, with their thin and thick parts being opposed to each-other relative to the center of the rod. However, when the washers in such a pair are in an “equal rotation” arrangement, with their thick parts being on the same side from the center, the rod will show a curvature at that point away from those thick parts of the washers in the amount of the sum of (or 2 times) the angles of the individual wedge shapes. If a full-length rod contains, for example, 12 segments with 11 interspersed pairs of washers, and if each wedge washer has a 2° wedge angle, then the rod can be curved by a maximum of 44°. By using partial rotation of the combined pair of washers, the curvature of the rod can follow or represent any desired shape in three dimensions. Additionally, limited elasticity of the whole Flex-Rod assembly can be provided by means of flat elastic washers between the segments or by anchoring of the connecting features (central wire) in an elastic manner, for example, by means of spring washers at their ends.

### CROSS REFERENCE TO RELATED APPLICATIONS

### General Background

Orthopedic deformations, specifically of the spine, call for supportive or corrective therapy. In case of a weekend and not severely-deformed spine (for example, after accidents or upon on-set of kyphosis), only retention of remaining shape or curvature may be necessary. However, if correction of curvature or shape of the spine is desired, adaptive force application to the spine is an indicated therapeutic intervention. It is known that temporarily forced adjustment of form can lead not only to short-term forced recovery of form but also to long-term natural and permanent recovery of form by means of bone or ligament adaptation or growth. Therefore, it is customary to implant various rods or other mechanical elements and attach those by means of screws or other fasteners to bone elements or the vertebrae of the spine (or the rib cage) in order to provide such forced adjustment of form. If such rods for implantation are available only in linear straight shapes, they may not be suitable for curvature applications. To adapt straight rods to desired curvatures is difficult. A rod implant is needed that can be easily curvature adapted in the operating room just prior to implantation and could be further adjusted in the course of time after implantation. This is what the proposed Flex-Rod provides. Similar applications may exist in general construction.

As known, a specific problem arises for fixed implants with the natural adjustment or growth of bone or spine segments. Not only should an optimal amount of pressure of the inserted element against the bone or spine be maintained, but, actually, an adjustment of curvature or shape of the implanted element may become necessary in order to follow progressing form or curvature adjustment and to continue exerting the desired pressure for further form or curvature adjustment.

The here-described invention “Flex-Rod, Curvature-Adaptable” provides the design of a rod which, after initial curvature adaptation, can supply the amount of support or pressure to the bone or spine as initially needed, however, as the bone or spine adapts or requires more or different support or pressure, the proposed Flex-Rod can be simply curvature or shape adapted even after implant, in three dimensions, and within a wide range (in contrast to existing rod designs that are permanently rigid and would have to be exchanged by incurring an additional and often complex invasive procedure).

A specific benefit of “curvature adaptability” lies in the great ease of curvature or shape formation of the proposed Flex-Rod as has to be done by the physician with great precision before implantation in the course of invasive procedures within the setting of an operating room. As an additional benefit, the curvature-adaptable Flex-Rod allows for the avoidance of additional surgical intervention for necessary curvature or shape adaptation as incremental form adjustment of the spine or natural growth occurs.

### SPECIFIC EXAMPLES OF APPLICATIONS

When curvature of the spine (scoliosis) occurs, quite often among still growing juveniles, an on-going forced adjustment of the spine toward a more natural curvature is the goal of the common surgical therapy. A different and progressive curvature of the spine (kyphosis), occurring more often among the elderly, but also among the young, may require, at the minimum and during its early stage, form retention of the spine to avoid further degradation of form. This may also be required after traumatic impact on the spine in an accident.

At present, solid rods, often several of them in sequence, attached to sequences of vertebrae by means of screws, are the common solution.

The problem with this traditional method results from the fact that, as the spine would respond by reducing the curvature or as the juvenile spine grows, surgical replacement of the solid rods would become necessary.

A preferable solution would consist of the use of a single, extended (long) and adaptively curved implant for the full length of the spine curvature to be treated. This would require the curvature adaptation of the supporting implant commensurate with the reduction or adjustment of the curvature of the arch of the spine in the course of time. Extended rods were not used in the past due to the difficulty in precisely adapting them to the desired curvature of the spine upon invasive surgery. Furthermore, curvature change would require the replacement of such an extended and fixed rod assembly by means of another, complex, invasive procedure.

The proposed “Flex-Rod, Curvature-Adaptable” solves these problems and offers an ideal new tool to the orthopedic surgeon for scoliosis, kyphosis, or accidental impact correction with minimal adaptation difficulty before or after implantation.

### SUMMARY OF THE INVENTION

### Objects

It is the object of this invention to provide a simple rod-shaped device—for example, as an implant for orthopedic applications in scoliosis or kyphosis, or in general structural applications—to provide retention or correction of form or curvature over extended sections of, for example, the spine by means of a single rod implant that can be easily curvature adapted in three dimensions over an extended length at the beginning of invasive procedures before implantation or, additionally and subsequently, at any time after implantation.

### DESCRIPTION OF THE DRAWING OF THE STRUCTURE OF THE INVENTION “FLEX-ROD, CURVATURE-ADAPTABLE”

FIG. 1 in Drawing 1/3 presents a schematic illustration of the invention with an arbitrarily selected length and an exaggerated diameter for ease of representation. The actual length of the proposed Flex-Rod could be as long as almost the full length of the spine, or any part thereof, extending above the hip region (sacral region), mainly along the thoracic region but, occasionally, also along the lumbar region. The Flex-Rod would have a round or oblong diameter, see the round cross section in FIG. 2 in Drawing 1/3, that, for stability or stiffness reason, would be as large as still tolerable for orthopedic implantation or the general structural case on hand.

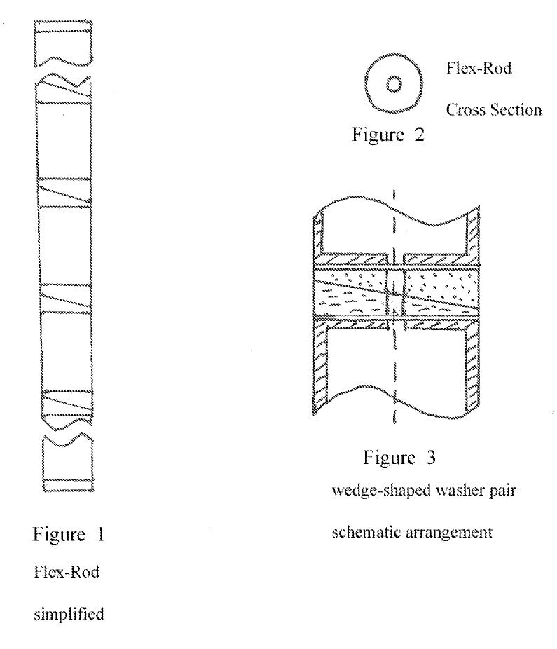

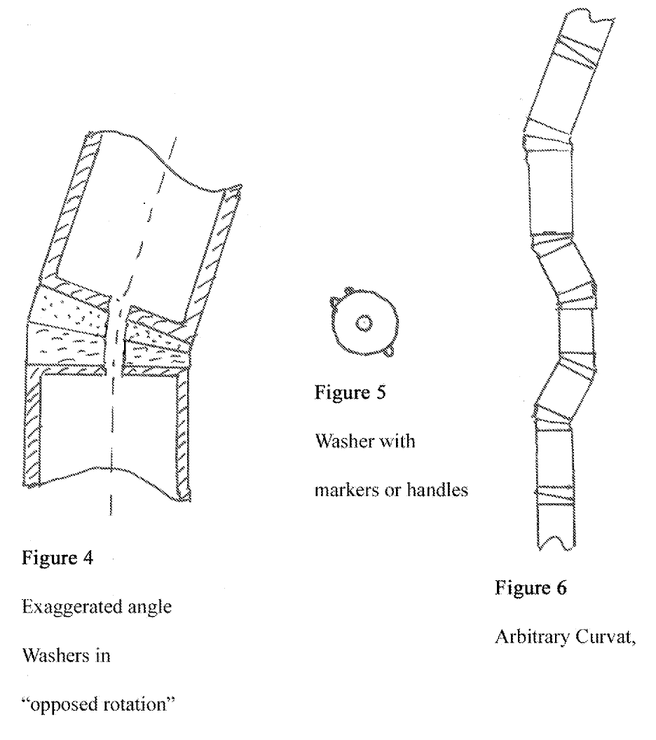

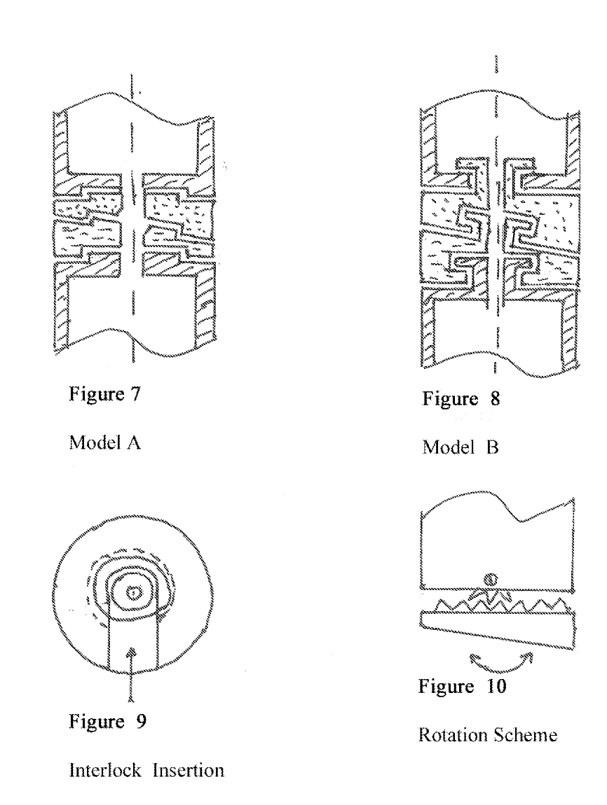

The “Flex-Rod”, as shown in FIG. 1 in Drawing 1/3, can contain a large number of segments, at least as many as the number of vertebrae being covered by the procedure. Between the segments, pairs of wedge-shaped washers are inserted, see FIG. 3 in Drawing 1/3. If preferred, these washers can be covered by flat elastic washers on one or both sides, see FIG. 3 in Drawing 1/3.

Description of the Function of the Wedge-Shaped Washers:

In FIG. 3 in Drawing 1/3, a pair of washers is shown inserted between two rod segments and arranged in an “opposed rotation” arrangement, with their thin and thick ends being 180 degrees opposed to each other relative to the center of the rod, whereby their wedge shapes cancel each other out and leave the rod in a linear, straight configuration. However, when the pair of wedge shaped washers is in an “equal rotation” arrangement, see FIG. 4 in Drawing 2/3, with their thick ends being on the same side of the rod, the wedge function of the two washers of the pair becomes cumulative and the rod will obtain a curvature of 2 times the angle of each of the washers, see FIG. 4 in Drawing 2/3. Since the angle of the wedge of the washers can be manufactured as needed, the incremental angle of rod curvature obtainable from each equal-rotated pair is selectable. Typically, the wedge angle would be selected between 0.5 and 5 degrees allowing for curvature insertion between 1.0 and 10 degrees at each rod segment. Multiple pairs of washers at an individual segment could accomplish larger curvature changes or subdivision into finer increments. In an extreme case, a curvature of the spine of 90° over 7 thoracic vertebrae (including 6 flex points between them) could be curvature-adapted by a Flex-Rod with groups of 3 pairs of washers at each flex point, each having a 2.5° wedge angle. This would allow for a three-step re-adjustment to a zero angle curvature or an unevenly distributed re-adjustment. If more pairs with smaller angles are used in each group, finer steps of adjustment become available. A small protrusion at one point or at opposed points of the periphery of the washers would allow for easy rotation and would serve as an indicator of the washer position, see FIG. 5 in Drawing 2/3. To prevent subsequent loss of rotation angle, simple dimples in the washer surfaces facing rod segments and in the rod segment surfaces facing washers would retain the rotation position of pairs of washers within a sufficiently close range. Undulated or ridge-and-valley treatment of the washer surfaces within a pair, in opposite manner, would allow for only “opposite rotated” or “equal rotated” arrangements of pairs to be stable.

Curvature of the Flex-Rod in Two or Three Dimensions:

FIG. 6 in Drawing 2/3 presents a schematic picture of a Flex-Rod with various curvatures in 2 dimensions. But it is obvious that a combined rotation of a pair of wedge-shaped washers by 90 degrees would allow a curvature of the rod in a plane at a 90 degree angle to the drawing paper or, in other words, in a third dimension. In a more general way, the pairs of washers can be jointly rotated in any desired angle and, subsequently, by going from “opposed rotation” to “equal rotation”, a curvature of the Flex-Rod can be obtained in any three-dimensional shape, allowing for the perfect adaptation of the Flex-Rod to the actual or desired shape or curvature of the spine or the structural problem on hand.

Connection Between the Washers and the Rod Segments and their Retention, “Model A”:

In this implementation of the Flex-Rod, the wedge shaped washers and the rod segments have connecting surfaces provided with a stepped profile in a cross section, see FIG. 7 in Drawing 3/3. In other words, their connecting surfaces are not entirely flat but, on their “top” surfaces, they are provided with a flat outer rim at one elevation and a more elevated, concentric, central surface or a smaller diameter rim in their central area. Inversely, their “lower” surfaces have also a flat outer rim but are provided with an inward-stepped concentric cavity or smaller diameter concentric groove in their central area. The terms “top” and “lower” relate to the FIG. 7 in Drawing 3/3. However, an inversion of the Flex-Rod would invert these terms. This stepped surface design of all washers and rod segments will allow the fitting of always the following next lower item of assembly into the preceding higher item of assembly—still allowing free rotation for the desired Flex-Rod curvature adaptation. A central hole in the covers of all washers and al rod segments allows for the stringing of a central wire, firmly or elastically attached to end caps of the rod, for corresponding stiffness or elasticity of the whole Flex-Rod assembly. Between the washers in a pair, the passage hole for the central wire may have partially flared openings where the washers of a pair connect in order to provide for smooth passage of the central element in case of the “equal rotation” arrangement.

Connection Between the Washers and the Rod Segments and their Retention, “Model B”:

An alternative interconnection to the stepped-surface-connection and central wire connectivity between elements of the Flex-Rod as shown in the cross section FIG. 7 in Drawing 3/3 can be accomplished by an interlocking design as, for example, shown in FIG. 8 in Drawing 3/3. There, the attachment of the wedge-shaped washer among themselves and to rod segments is shown. Therefore, the thickness of the washers would have to be larger than in a “Model A” Flex-Rod in order to accommodate the interlocking feature, as indicated in FIG. 8 in Drawing 3/3. The assembly of the Flex-Rod is accomplished by sliding each element of the rod into the preceding one by sliding the locking feature through a slit on the side of the preceding element. A top view of the locking area between two elements is shown in FIG. 9 in Drawing 3/3. The locking surface in the receiving element is not complete but incorporates a slit to let the stem of the locking feature of the following element pass into the central position. The parts will be kept from separating again by the central element (wire) subsequently strung through their centers. The initial installation of the central element (wire) is facilitated by providing a biologically inert (to prevent interaction with body fluids) filler for the somewhat longer rod segments, drilled from end to end to provide for guided passage of the central element upon installation.

Curvature Adaptation of the Flex-Rod after Implantation, Self-Adaptive:

Different methods of curvature adaptations of the Flex-Rod in either a self-adaptive (automatic) mode or under external control can be proposed and may become the subject of future patents. A few shall be mentioned here as examples and to become part of this patent application. Self-adaptive adjustment: As explained, the curvature adjustment of a segment of the Flex-Rod occurs through change of orientation of only one of the two wedge-shaped washers in a pair from “opposed rotation” arrangement to “equal rotation” arrangement, see the preceding paragraph [0014], with both arrangements being stable but not those in any other intermediate angle arrangement between them. A ligament could be attached to the spine at a critical point such that flexing of the spine under certain spine shape conditions would result in a flipping of one of the washers in a pair from “equal rotation” to “opposed rotation” arrangement, especially if the pair was spring loaded, thereby changing the curvature of the Flex-Rod. A more advanced design may, in the future, incorporate miniature motors under the control of spine conditions to accomplish washer rotations.

Curvature Adaptation of the Flex-Rod after Implantation, Under External Control:

While an implantation of a Flex-Rod would still be in process, the attending surgeon may turn any one or any combination of all the wedge-shaped washers of a Flex-Rod by hand, thereby changing the curvature of the Flex-Rod into the desired shape. Once the Flex-Rod is fully implanted and the area of surgery closed and healed, rotation of the washer for further curvature adaptation of the rod becomes more of a problem. As indicated in the preceding paragraph [0018], future designs of the Flex-Rod may incorporate miniature motors under external control to accomplish the desired washer rotations. A more direct approach consists of providing each critical washer interface with a miniature screw and a mechanical design of the washer surface as, for example, shown in FIG. 10 in Drawing 3/3. The miniature screw would be incorporated in the bottom area of a rod segment (that would not rotate) and would interact with a serrated rim on the upper washer surface, thereby providing for the rotation of the washer by means of the inserted screwdriver.

1. A patent is claimed for a mechanical support rod which is of linear, straight shape when not adapted, but which can be adapted to any three-dimensional curvature or shape within a wide range by means of wedge-shaped washers inserted between rod segments.

2. A rod design as described by Principal claim # 1 further including that it can be surgically implanted and attached to the spine or any other bone or can be used in general construction, however is not of permanently fixed structure but is curvature-adaptable within a wide range by means of wedge-shaped washer inserted between rod segments.

3. A rod design as described by Principal claim # 1 further including that, in case of the usage of the rod as an orthopedic implant to retain or modify spine curvature, it is subdivided into segments that can correspond to the vertebrae and where the wedge-shaped washer between the rod segments result in the desired angle of the rod at those points to arrive at an overall curvature of the rod as desired for correct spine support or desired spine curvature correction.

4. A rod design as described by Principal claim # 1 further including a design where the wedge-shaped washers located between the rod segments are assembled permanently within the rod assembly and always in pairs or in multiples of pairs, resulting in zero curvature correction of the rod at those points where the washers within a pair are assembled in an “opposed rotation” arrangement, with their thin and thick parts being 180 degrees opposed to each other within the assembly and, thereby, canceling each other out, but resulting in a curvature correction of the rod of 2 times the angle of the wedge of the washers when assembled in an “equal rotation” assembly, with their thin and thick parts respectively being located exactly on the same side of the center of the rod, whereby accumulation of angle occurs from the addition of the two thick parts of the wedges.

5. A rod design as described by Principal claim # 1 further including that multiples of pairs of wedge-shaped washers may be assembled between rod segments to allow for differentiated angle adjustments between rod segments.

6. A rod design as described by Principal claim # 1 further including that the wedge-shaped washers can be rotated, not only individually to allow the conversion from “opposed rotation” arrangement to “equal rotation” arrangement but as a pair, allowing for curvature of the rod in any direction, thereby permitting the formation of any three-dimensional curvature or form of the rod within wide margins.

7. A rod design as described by Principal claim # 1 further including that the surfaces of the segments and washers can be provided with dimples, ridge-and-valley features, or other retention features to still permit the rotation of the washers, but to prevent them from too easily changing their rotation angle.

8. A rod design as described by Principal claim # 1 further including that the surfaces of the segments and washers can be inserted into each other by means of stepped surface features as, for example, concentric plateaus or rims and their opposed opposites.

9. A rod design as described by Principal claim # 1 further including inserted washers between the segments where the whole assembly may be kept together by means of a central wire that can have a spring-loaded suspension at the end-caps of the rod to provide for selectable and limited elasticity of the rod.

10. A rod design as described by Principal claim # 1 further including inserted washers between the rod segments where the parts of the assembly may be kept together by means of locking features protruding from the surfaces of each element of the rod and locking into opposite elements in the next following rod assembly.

11. A rod design as described by Principal claim # 1 further including interlocking features between the rod elements which, upon assembly, slide sidewise into the preceding element through slits and are kept from moving out again by means of a central assembly element as, for example, a wire running through the center of the whole rod assembly.

12. A rod design as described by Principal claim # 1 further including that additional elasticity of the rod is accomplished by adding flat, elastic washers to the stacks of pairs of wedge-shaped washers.

13. A rod design as described by Principal claim # 1 further including that curvature correction of the rod is accomplished by rotation of the washers by means of serrated ridges on the top surface of the top washers and by screws imbedded in the lower portion of the segments next to the top washer and accessible from the outside such that, when the screws are turned, the washers are being rotated.

14. A rod design as described by Principal claim # 1 further including that the rotation of the washers is accomplished by sub-miniature motors under external or adaptive internal control.

15. A rod design as described by Principal claim # 1 further including that the rotation of the washers is accomplished by mechanical coupling to, in case of orthopedic applications, critical points of the spine or bone or, in case of general structural applications, to any other critical point of the structure, such that rotation is put under adaptive internal control.

16. A rod design as described by Principal claim # 1 further including such pairs of washers between the segments where the pairs are spring loaded such that the rotation of one washer relative to the other within a pair can be triggered and is sudden and fully between either “opposed rotation” and “equal rotation” arrangement or in the opposite direction either under external control or, in self-adaptation, under internal control.

17. A rod design as described by Principal claim # 1 further including that the design of the washers is such that one can visually determine the rotation position of each washer by means of protruding markers at their thin or thick ends.
